(19) United States
(12) Patent Application Publication
(10) Pub. No.: US 2023/0120438 A1
(43) Pub. Date: Apr. 20, 2023

(54) OTOSCOPE

(71) Applicant: HEINE Optotechnik GmbH & Co. KG, Gilching (DE)

(72) Inventors: Bernd KABBECK, Gilching (DE); Timo MARTIN, Gilching (DE); Felix MICHEL, Gilching (DE)

(21) Appl. No.: 17/966,215

(22) Filed: Oct. 14, 2022

(30) Foreign Application Priority Data
Oct. 18, 2021 (DE) 10 2021 126 955.5

(51) Int. Cl. A61B 1/227; A61B 1/07; A61B 5/00; A61B 1/06

(52) U.S. Cl. CPC A61B 1/227; A61B 1/07; A61B 5/0077; A61B 1/0607

(57) ABSTRACT

An otoscope including a head, a holder for an ear funnel, a primary lighting unit and a secondary lighting unit. The primary lighting unit is configured for examining the ear and the secondary lighting unit is configured for examining a part of the body that differs from the ear.

### FIELD OF THE DISCLOSURE

The disclosure relates to an otoscope.

### BACKGROUND

Otoscopes for examining the ear, in particular the tympanic membrane have been known for over 90 years. Typically, such otoscopes comprise a holder for a removable ear funnel as well as an optical system using magnification whose beam path is directed through a central opening of the ear funnel. In this way, it is possible to examine the tympanic membrane or another part of the ear.

In addition, it is known to provide otoscopes with a lighting unit to enable for the examination of the ear the optimal illumination of the examination area in front of the holder for the ear funnel.

However, examinations of other parts of the body using an otoscope are not possible without risk.

### SUMMARY

Therefore, an otoscope that enables examinations of other parts of the body besides the ear is needed.

Thus, there is provided an otoscope comprising a head, a holder for an ear funnel, a primary lighting unit and a secondary lighting unit. The primary lighting unit is configured for examining the ear and the secondary lighting unit is configured for examining a part of the body that differs from the ear.

Owing to the secondary lighting unit that is configured and optimized for examining a part of the body that differs from the ear, examinations of other parts of the body besides the ear, for example the eye, skin and/or the mouth and throat, are now possible without any risk.

The specific configuration for examining parts of the body with the primary and secondary lighting unit can relate to an illuminated examination area and/or to light properties, such as wavelength, color temperature, polarization, beam geometry, maximum brightness and suchlike.

In particular, the beam directions of the primary and secondary lighting unit extend from the patient side of the head towards the patient.

For example, the beam path of the secondary lighting unit does not run through a central opening of the holder and/or runs completely external to the holder.

For example, the secondary lighting unit is located completely external to the holder and/or ear funnel.

For example, the holder starts from the head and comprises a central opening.

In one aspect, the otoscope defines a primary examination area and the primary lighting unit is configured to illuminate the primary examination area, in particular wherein the secondary lighting unit is configured to illuminate a secondary examination area that differs from the primary examination area. As a result, the different examination areas for different parts of the body to be examined are defined and the lighting unit are therefore better configured for the corresponding parts of the body to be examined.

In particular, the primary and secondary examination areas are completely different. For example, the primary examination area extends 1 cm to 3 cm in front of the central opening of the holder.

The primary examination area can be located axially in front of the central opening.

In an embodiment, the otoscope comprises an optical system comprising a lens and/or a camera, wherein the beam path of the optical system runs through the central opening of the holder, in particular wherein the primary examination area corresponds to a region around the focal point of the optical system with the axial length of the depth of field of the optical system, thereby enabling a simple examination of the ear.

The primary examination area can be expanded as large as the central opening in the radial direction relative to the central opening.

For simpler and safer handling, the holder can extend from the head in a funnel shape and/or can comprise an attachment means for attaching an ear funnel on its external side.

In an embodiment, the primary lighting unit emits light with primary light properties and the secondary lighting unit emits light with at least in part other light properties than the primary light properties, in particular wherein the light properties include wavelength, color temperature, maximum brightness, beam geometry and/or polarization. In this way, the light properties can also be configured according to the part of the body to be examined, thereby optimizing the quality of the lighting for the respective purpose.

For example, the light emitted by the primary lighting unit has a wavelength ranging from 200 nm to 1000 nm, in particular from 380 nm to 780 nm, a color temperature ranging from 3000 K to 4500 K, a maximum brightness ranging from 5 lm to 20 lm and/or no polarization, in particular wherein the secondary lighting unit emits light with a wavelength, a color temperature and/or a brightness at least in part outside the corresponding value ranges of the primary lighting unit and/or emits polarized light. In this way, the lighting can be adjusted for each part of the body to be examined.

To optimize the lighting further, the light emitted by the primary lighting unit can have a beam geometry that differs from the beam geometry of the light emitted by the secondary lighting unit.

In another aspect, the head has a handle side, wherein the secondary lighting unit is located on the head on the side of the holder facing away from the handle side. Owing to this arrangement, the user must not change their grasp on the otoscope if another part of the body other than the ear is to be examined.

To adjust the light properties further, the secondary lighting unit can comprise at least one secondary light source and one optical element, in particular a lens, an aperture and/or a filter.

The optical element is in particular an element that is separate from the secondary light source.

The optical element can be a spherical lens.

In particular, the beam path of the secondary lighting unit does not run through the central opening of the holder and/or runs completely external to the holder.

In an embodiment, one of at least one optical element is a lens and/or an aperture that is located in the beam path of the secondary light source, in particular wherein the lens or the aperture is moveable relative to the secondary light source along the optical axis of the secondary lighting unit. As a result, the light properties of the secondary lighting unit can be changed easily.

In an embodiment, the secondary lighting unit comprises a moveable support in which various optical elements of the secondary lighting unit are located and/or provided, in particular various optical elements of the same type, wherein the moveable support runs and can be moved in such a way through the beam path of the secondary light source that said one of the optical elements of the support which is in the beam path can be changed. By means of the various optical elements of the support, the light properties of the secondary lighting unit can be adjusted over an extremely wide range.

For example, the support is a ring or annular section, wherein the ring or annular section is configured concentrically to the central opening of the holder and is configured rotatably around the axis of the central opening, wherein the optical axes of the optical elements run parallel to the axis of the central opening, thereby enabling a particularly large number of different optical elements on the support.

Alternatively or additionally, the support is a ring or annular section, wherein the ring or annular section is configured rotatably about a rotational axis that is perpendicular to the axis of the central opening, wherein the optical axes of the optical elements run perpendicular to the rotational axis, thereby ensuring that the configuration of the support saves space.

In an embodiment, the secondary lighting unit comprises several secondary light sources and/or several assemblies comprising at least one secondary light source and one optical element, wherein the light sources or the assemblies emit light with light properties that differ from each other and/or wherein the light sources and/or the assemblies are located on the head around the holder. In this way, the light properties of the light emitted by the different assemblies can be optimized in each case for examining different parts of the body.

The light sources of different assemblies can be different types, for example LEDs or laser diodes.

For simple handling, the otoscope can have a handle on which the head is attached, in particular wherein at least one control element is provided on the handle, with said control element being capable of controlling the primary and/or secondary lighting unit.

### BRIEF DESCRIPTION OF THE DRAWINGS

Additional features and advantages of the disclosure are found in the following description as well as the attached drawings to which reference is made. In the drawings:

FIG. 1 shows an otoscope according to the disclosure in a perspective view from behind,

FIG. 2 shows the otoscope according to FIG. 1 in a perspective view on the head from the front,

FIG. 3 shows a sectional view through the otoscope according to FIG. 1,

FIG. 4 shows a sectional view through a second embodiment of an otoscope according to the disclosure,

FIG. 5 shows a front view of a head of the otoscope according to the disclosure in accordance with a third embodiment,

FIG. 6 shows a support for optical elements of the otoscope according to FIG. 5, and

FIG. 7 shows a sectional view through an otoscope according to the disclosure in accordance with a fourth embodiment.

### DETAILED DESCRIPTION

Lists having a plurality of alternatives connected by “and/or”, for example “A, B and/or C” are to be understood to disclose an arbitrary combination of the alternatives, i.e. the lists are to be read as “A and/or B and/or C”. The same holds true for listings with more than three items.

In FIG. 1, an otoscope 10 is shown in a perspective view from behind, thus from that side that faces the user, for example the physician, when used correctly.

Within the context of this disclosure, this side is therefore termed the “user side”. The opposing side is termed the “patient side”.

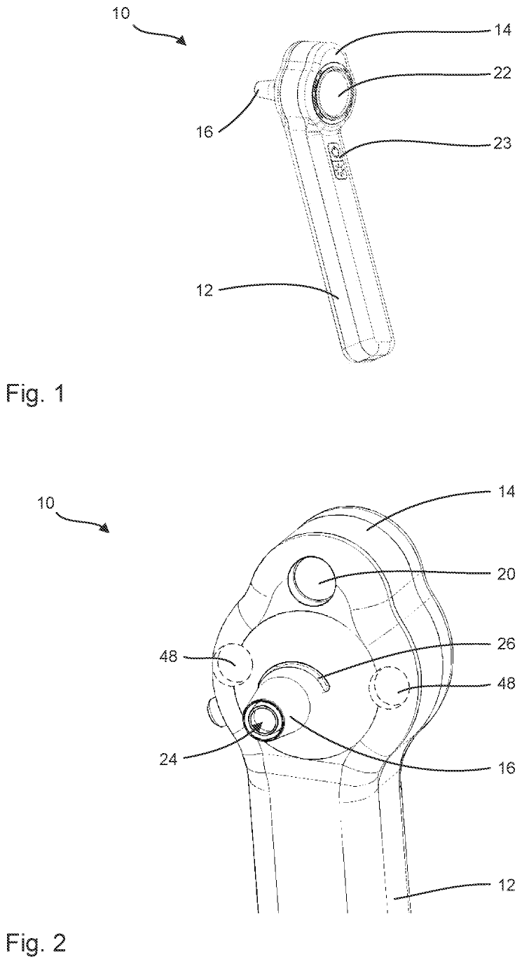

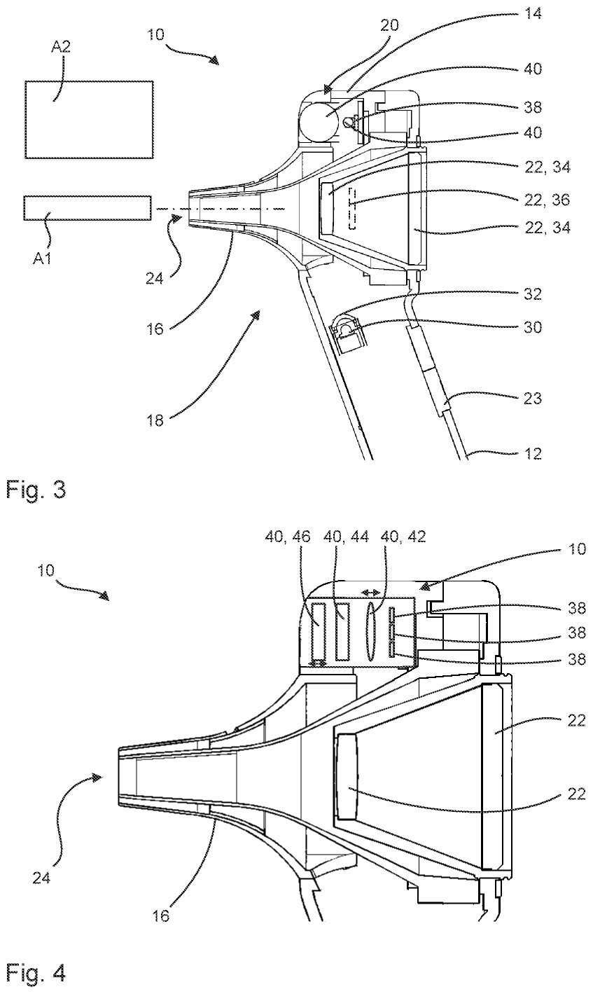

The otoscope 10 comprises a handle 12, a head 14, a holder 16 for an ear funnel, a primary lighting unit 18 (FIG. 3), a secondary lighting unit (FIG. 2), an optical system 22 as well as two control elements 23.

Within the scope of this disclosure, the terms “primary” and “secondary” are used to distinguish between different parts and do not indicate a minimum number or hierarchies between the parts.

The control elements 23 are located on the handle 12 in the shown embodiment and are, for example, push buttons.

By means of the control elements 23, the primary lighting unit 18 and/or the secondary lighting unit 20 can be operated or controlled.

The head 14 of the otoscope 10 is shown in FIGS. 2 and 3 in a perspective view from the front and in a sectional view, respectively.

The holder 16 is located on the head 14, for example extending away from the head 14 as a single piece.

The holder 16 has a funnel shape and tapers away from the head 14 towards the point that is furthest away from the head 14, at said point the central opening 24 is configured.

On the external side, the holder 16 comprises an attachment means 26 for attaching an ear funnel (not shown) in a known way.

The holder 16 is configured to be double walled, as can be seen in FIG. 3, wherein the two walls of the holder 16 run concentrically about the axis of the central opening 24, however with different radii.

In the area of the central opening 24, an annular gap is therefore formed between the two walls.

The area between both walls and thus also the annular gap forms a part of the primary lighting unit 18. In particular, the double wall and the resulting annular gap correspond to an annular aperture.

In addition, the primary lighting unit 18 comprises a primary light source 30 and optionally an optical element 32, in the shown embodiment a lens.

In addition, the primary lighting unit 18 can comprise a bundle of optical fibers that is not shown in the Figures in order to improve the overview. The optical fiber bundle runs from the primary lighting unit 18 and the optical element 32 between both walls of the holder 16 to the annular gap.

The primary light source 30 is configured, for example, to emit light with specific light properties. For example, the primary light source 30 is an LED.

The light properties can include the wavelength, for example the mean wavelength, the color temperature, the maximum brightness and/or the polarization.

The primary light source 30 and thus as a result the primary lighting unit 18 emit light that is optimal for the examination of the ear, for example unpolarized light with a wavelength between 200 nm and 1000 nm, in particular between 380 nm and 780 nm, a color temperature ranging from 3000 K to 4500 K and a maximum brightness ranging from 5 lm to 20 lm.

The optical element 32 is a lens in the shown embodiment, however, it can also be a filter, such as a wavelength filter or a polarization filter. Even the annular gap at the central opening is to be regarded as an optical element 32 of the primary lighting unit 18.

The optical system 22 is configured on the head 14 and at least in part in the head 14 and has a beam path that extends through the head 14, more specifically from the user side (right-hand side in FIG. 3) to the patient side (left-hand side in FIG. 3). The beam path runs within the inner wall of the holder 16.

In the shown embodiment, the optical system 22 comprises two lenses that define a primary examination area A1, wherein one of the lenses 34 is attached in the head 14 and the other one of the lenses 34 is attached on the user side of the head 14.

The primary examination area A1 corresponds, for example, to a region in the axial direction of the central opening 24 and in the direction of the beam path of the optical system 22 in front of the central opening 24 that extends around the focal point of the optical system 22. The primary examination area A1 can have an axial length here that corresponds to the depth of field of the optical system 22. In the radial direction, the primary examination area A1 is approximately as large as the central opening 24.

Instead of one or both lenses 34, a camera 36 (indicated by dashed lines in FIG. 3) can also be provided in the optical system 22.

The primary examination area A1 is illuminated during an examination of the ear by means of the primary lighting unit 18.

During operation of the primary lighting unit 18, light is thus emitted by the primary light source 30 with the light properties and then passes through said one or more optical elements 32, thereby enabling the light properties to be changed, such as the polarization. The light is then coupled into the optical fibers of the optical fiber bundle and runs in the optical fiber bundle between both walls of the holder 16 to the central opening 24 where finally exits the otoscope 10.

Upon exiting through the central opening 24, the light of the primary lighting unit 18 has primary light properties that also include beam geometry that in this case is annular.

In this way, the primary examination area A1 is optimally illuminated by the primary lighting unit 18. The primary lighting unit 18 is thus configured to illuminate the primary examination area A1.

The primary examination area A1 extends, for example, between 1 cm and 3 cm in the axial direction in front of the central opening 24 of the holder 16.

The secondary lighting unit 20 is also located in the head 14 of the otoscope 10.

In contrast to the primary lighting unit 18, the secondary lighting unit 20 is configured and optimized for examining a part of the body that differs from the ear.

The secondary lighting unit 20 is located, for example, above the optical system 22 and the holder 16, i.e. on the side of the head 14 facing away from the handle 12 and the handle side.

The secondary lighting unit 20 is thus configured completely external to the holder 16 and/or the ear funnel.

The secondary lighting unit 20 illuminates a secondary examination area A2 that differs from the primary examination area A1 or differs from it in part. In particular, there are no overlaps between the primary examination area A1 and the secondary examination area A2 or the area of overlapping is less than 20% of the total area of the primary examination area A1.

The secondary examination area A2 is located in front of the same side of the head 14 as the primary examination area A1, thus on the patient side of the head 14.

Accordingly, the beam directions of the primary lighting unit 18 and the secondary lighting unit 20 are on the same side of the head 14.

The beam path of the secondary lighting unit 20 runs completely external to the holder 16, in particular not through the central opening 24 of the holder 16.

The secondary lighting unit 20 is designed, for example, in a cavity of the head 14 and comprises a secondary light source 38 as well as at least one optical element 40.

In the shown first embodiment according to FIG. 3, the secondary lighting unit 20 comprises two optical elements 40 in the form of lenses, for example spherical lenses.

The optical elements 40 are elements that are separate from the secondary light source 38.

The use of a single spherical lens is also conceivable.

The secondary light source 38 emits light with specific light properties, wherein the light runs through the optical elements 40 before illuminating the secondary examination area A2. The secondary light source 38 is for example an LED or a laser.

In particular, the beam path of the secondary lighting unit 38 does not run through the central opening 24 of the holder 16 and/or runs completely external to the holder 16.

The secondary light properties of the light emitted by the secondary lighting unit 20 differ at least in part from the primary light properties of the primary lighting unit 18 in the primary examination area A1. In particular, the values of the secondary light properties are least in part outside the corresponding value ranges of the primary light properties.

For example, the light emitted by the secondary lighting unit 20 is configured for examining the eye and therefore comprises another wavelength range, another color temperature, another maximum brightness and/or another polarization.

In particular, the beam geometry of the light emitted by the secondary lighting unit 20 differs from the annular beam geometry of the primary lighting unit 18.

A user, in particular a physician, can use the otoscope 10 in the known way to examine the ear by placing an ear funnel on the holder 16 and then introducing this ear funnel into the ear of the patient. The primary lighting unit 18 is activated and the user examines the ear with the aid of the optical system 22.

If the user would like to now examine another part of the body, for example the eye, the user deactivates the primary lighting unit 18 and activates the secondary lighting unit 20. The user can do this by means of the control elements 23.

Owing to the configuration of the secondary lighting unit 20 for an examination of the eye, the user can perform an optimal examination without changing instruments.

FIGS. 4 to 7 show further embodiments of the otoscope according to the disclosure which substantially correspond to the first embodiment. Therefore, only the differences are discussed hereinafter and the same parts and parts with the same function are provided with the same reference signs.

In FIG. 4, a section is shown through the head 14 of the otoscope 10 that is similar to that of FIG. 3.

In this second embodiment, the secondary lighting unit 20 is configured differently.

Firstly, the secondary lighting unit 20 comprises three secondary light sources 38 that in particular can emit light with different light properties or are even different types of light sources (LED, laser).

In addition, three optical elements 40 are provided, namely a lens 42, a filter 44 as well as an aperture 46, that are located in the beam path of the secondary light source 38.

By means of the optical elements 40, the secondary light properties of the light emitted by the secondary lighting unit 20 can be adjusted further.

One or several of the optical elements 40, in particular the aperture 46 and/or the lens 42 can be attached moveably so that they can be moved linearly in the direction of the beam path relative to the secondary light source 38, as is indicated by the arrows in FIG. 4. The beam geometry of the secondary lighting unit 20 can be adjusted through sliding.

It is conceivable that the filter 44 is a polarization filter or the aperture 46 is a slit aperture.

The optical elements 40 and at least one secondary light source 38 form an assembly 48. It is conceivable that the secondary lighting unit 20 comprises a variety of these assemblies 48 from a secondary light source 38 and corresponding optical elements 40 to enable the provision of optimized light for different examinations of parts of the body other than the ear.

For example, each of the assemblies illuminate the secondary examination area A2.

The various assemblies 48 can be located on the head 14 at least in part around the holder 16, as is indicated in FIG. 2 by means of the dashed circles.

For example, the emitted light of one of the assemblies 48 can be configured or optimized for examining the eye and the light of another assembly 48 for examining the mouth and throat.

In particular, the secondary light source 38 of the different assemblies 48 can be different. Thus, an assembly 48 can comprise a laser diode and structured aperture as secondary light source 38 in order to provide what is termed structured light in the secondary examination area A2.

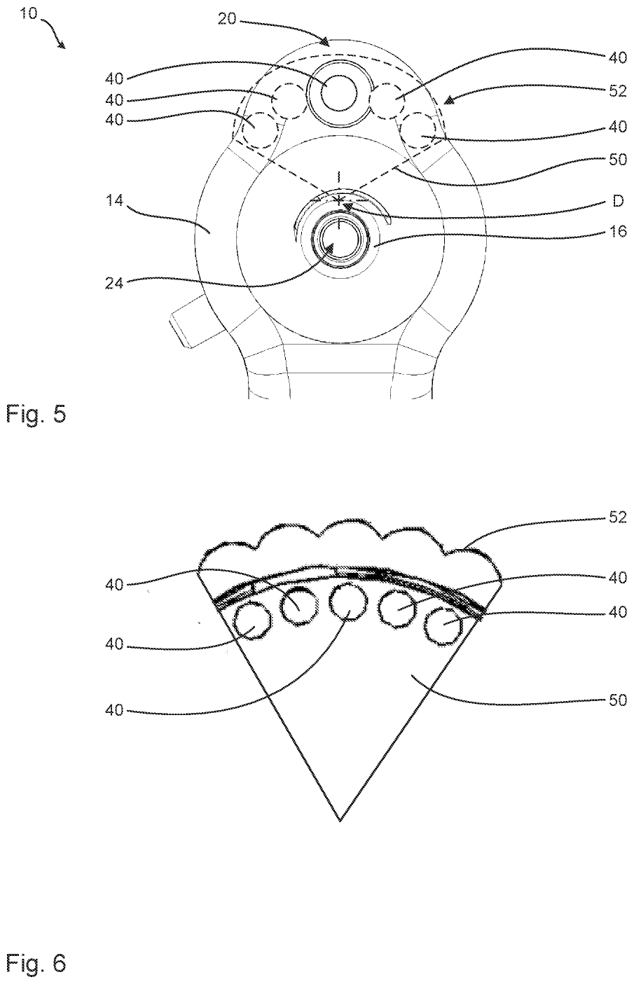

The FIGS. 5 and 6 show a third embodiment of the otoscope 10 according to the disclosure.

The third embodiment substantially corresponds to the second embodiment according to FIG. 4, wherein one of the optical elements 40 of the secondary lighting unit 20 can be changed.

To this end, the otoscope 10 comprises a support 50 in the head 14, on which or in which different optical elements 40 of the same type can be configured. For example, the support 50 comprises different lenses 42, different filters 44 or different apertures 46. In FIG. 6, such a support 50 is shown exemplarily.

The support 50 can be designed as a ring or—as shown—as an annular section, wherein the optical elements 40 are located next to each other in the circumferential direction. In particular, the optical elements 40 have the same distance to the center point of the ring.

To this end, the optical axes of the optical elements 40 all comprise the same direction, namely perpendicular to the plane of the support 50.

The radially outer surface of the support 50 can comprise an actuation contour 52 to twist the support 50 manually.

As indicated in FIG. 5, the support 50 runs in the beam path of the secondary light source 38 so that one of the optical elements 40 is located in the beam path.

The support 50 is mounted rotatably in the head 14 about a rotational axis D, wherein the actuation contour 52 of the support 50 projects from the head 14 for manual actuation. The rotational axis runs parallel to the axis of the central opening 24 of the holder 16, in particular the rotational axis D corresponds to the axis of the central opening 24.

By rotating the support 50, that optical element 40 can be changed that is located in the beam path of the secondary light source 38. In this way, the light properties of the light emitted by the secondary lighting unit 20 can be changed to adjust these to the desired treatment situation or the part of the body to be examined.

Alternatively or in addition to manual actuation, an automatic rotation, for example an electrically powered rotation, of the support 50 is conceivable.

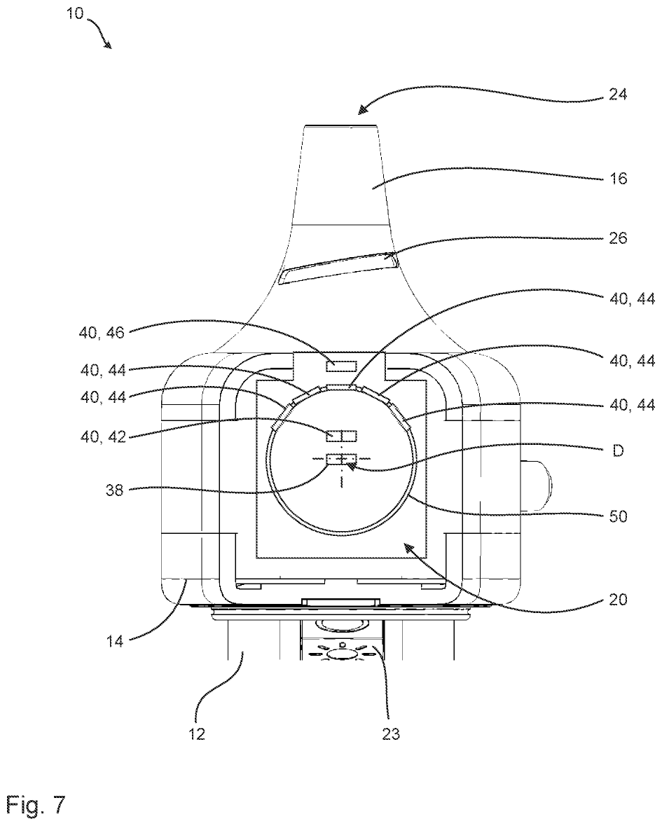

In FIG. 7, a fourth embodiment of the otoscope 10 according to the disclosure is shown. FIG. 7 shows a horizontal section through the head 14 of the otoscope 10 through the secondary lighting unit 20 above the holder 16 and the optical system 22.

The secondary lighting unit 20 of the fourth embodiment substantially corresponds to that of the second embodiment according to FIG. 4.

In addition, a support 50 is provided in the fourth embodiment, similar to the one described in the third embodiment, on which different optical elements 40 of the same type are located in order to change the optical elements in the beam path.

In the fourth embodiment, the support 50 is a ring, however it could just as well be an annular section or a flat slider.

The support 50 is configured rotatably relative to the head 14 about a rotational axis D that extends perpendicularly to the beam path or to the axis of the central opening 24, for example upwards. The support 50 is located around the secondary light source 38 and, if applicable, also around an optical element 40. The optical axes of the optical elements 40 of the support 50 point here in the radial direction of the support 50.

By rotating the support 50, the optical element 40 that is located in the beam path can also be changed in this embodiment. Also through this, the light properties of the secondary lighting unit 20 can be adjusted.

If the support 50 is a slider, the support 50 can move linearly perpendicular to the beam path.

Also in this embodiment, the rotation or movement of the support 50 can occur manually or automatically, for example by means of an electric motor.

It is conceivable that the secondary lighting unit 20, in particular the light properties, can be selected or controlled by means of the control elements 23.

The different features of the different embodiments can be combined with each other. In particular, it is conceivable that the second, third and fourth embodiments (FIGS. 4, 5, and 7) are combined with each other, wherein an optical element 40, for example the aperture 46, can be changed by means of a support 50, as described for the third embodiment (FIG. 5), and a further optical element 40, for example a filter 44 or a lens 42, can be changed by means of a support 50, as described for the fourth embodiment (FIG. 7).

It is also conceivable that several supports 50 of the same type are used to enable the changing of different optical elements 40.

1. An otoscope with a head, a holder for an ear funnel, a primary lighting means and a secondary lighting unit, wherein the primary lighting unit is configured for examining the ear and the secondary lighting unit is configured for examining a part of a body that differs from the ear.

2. The otoscope according to claim 1, wherein the otoscope defines a primary examination area and the primary lighting unit is configured to illuminate the primary examination area.

3. The otoscope according to claim 2, wherein the secondary lighting unit is configured to illuminate a secondary examination area that differs from the primary examination area.

4. The otoscope according to claim 1, wherein the otoscope comprises an optical system comprising at least one of at least one lens or one camera, wherein a beam path of the optical system runs through a central opening of the holder.

5. The otoscope according to claim 1, wherein the holder at least one of extends from the head in a funnel shape or comprises an attachment means for attaching the ear funnel on its external side.

6. The otoscope according to claim 1, wherein the primary lighting unit emits light with primary light properties and the secondary lighting unit emits light with at least in part other light properties than the primary light properties.

7. The otoscope according to claim 1, wherein the light emitted by the primary lighting unit has at least one of a wavelength ranging from 200 nm to 1000 nm, a color temperature ranging from 3000 K to 4500 K, a maximum brightness ranging from 5 lm to 20 lm, or no polarization.

8. The otoscope according to claim 7, wherein the secondary lighting unit at least one of emits light with at least one of a wavelength, a color temperature, or a brightness at least in part outside corresponding value ranges of the primary lighting unit or emits polarized light.

9. The otoscope according to claim 1, wherein the light emitted by the primary lighting unit has a beam geometry that differs from the beam geometry of the light emitted by the secondary lighting unit.

10. The otoscope according to claim 1, wherein the head has a handle side, wherein the secondary lighting unit is located on the head on the side of the holder facing away from the handle side.

11. The otoscope according to claim 1, wherein the secondary lighting unit comprises at least one secondary light source and one optical element.

12. The otoscope according to claim 11, wherein the optical element is at least one of a lens, an aperture or a filter.

13. The otoscope according to claim 11, wherein the one of at least one optical element is at least one of a lens or an aperture that is located in a beam path of the secondary light source.

14. The otoscope according to claim 13, wherein the lens or the aperture is moveable relative to the secondary light source along an optical axis of the secondary lighting unit.

15. The otoscope according to claim 11, wherein the secondary lighting unit comprises a moveable support in which various optical elements of the secondary lighting unit are at least one of located or provided, wherein the moveable support runs and is moveable in such a way through a beam path of the secondary light source that said one of the optical elements of the support which is in the beam path can be changed.

16. The otoscope according to claim 15, wherein the support is a ring or annular section, wherein the ring or annular section is configured concentrically to a central opening of the holder and is configured rotatably around an axis of the central opening, wherein optical axes of the optical elements run parallel to the axis of the central opening.

17. The otoscope according to claim 15, wherein the support is a ring or annular section, wherein the ring or the annular section is configured rotatably about a rotational axis that is perpendicular to the axis of a central opening, wherein optical axes of the optical elements run perpendicular to the rotational axis.

18. The otoscope according to claim 1, wherein the secondary lighting unit comprises several secondary light sources, wherein at least one of the light sources emit light with light properties that differ from each other or the secondary light sources are located on the head around the holder.

19. The otoscope according to claim 1, wherein the secondary lighting unit comprises several assemblies comprising at least one secondary light source and one optical element, wherein at least one of the assemblies emit light with light properties that differ from each other or the assemblies are located on the head around the holder.

20. The otoscope according to claim 1, wherein the otoscope has a handle on which the head is attached.
